(19) United States
(12) Patent Application Publication
(10) Pub. No.: US 2009/0246231 A1
(43) Pub. Date: Oct. 1, 2009

(54) Formulations containing borojo

(76) Inventors: Giovanni Valencia, Secaucus, NJ (US); Dan M. DeLaRosa, Coral Springs, FL (US)

Correspondence Address: Dan M. DeLaRosa, Suite 24C, 300 E. 77th Street, New York, NY, 10021, US

(21) Appl. No.: 12/381,652

(22) Filed: Mar. 16, 2009

Related U.S. Application Data
(60) Provisional application No. 61/070,562, filed on Mar. 25, 2008.

(51) Int. Cl. A61K 9/00; A61K 36/74; A61K 36/28; A61K 36/00

(52) U.S. Cl. 424/400; 424/769; 424/764; 424/776

(57) ABSTRACT

The present invention relates to a cosmetic and skin care formulation comprising: borojo; and at least one humectant; and at least one emollient. The present invention also provides for a dietary and nutritional supplement comprising: borojo; at least one mineral; at least one vitamin and at least one protein.

### RELATED APPLICATION

This application is related to U.S. Provisional Application Ser. No. 61/070,562, entitled “Formulations Containing Borojo” which was filed on Mar. 25, 2008.

### BACKGROUND OF THE INVENTION

1. Field of the Invention

This invention relates to cosmetic and skin care formulations and related methods of manufacture and more particularly, a cosmetic and skin care formulation containing borojo. In another embodiment, this invention relates to nutritional and dietary supplement formulations and related methods of manufacture and more particularly, a nutritional and/or dietary supplement formulation comprising borojo, minerals, vitamins, proteins, peptides and/or anti-oxidants.

2. Description of the Related Art

Borojo is a fruit that grows in the Amazon and Central America. Borojo has a high source of nutrients and has been used as a beverage and consumed orally. The present invention relates to the use of borojo for topical applications. In another embodiment, the present invention relates to the use of borojo with other nutrients to form nutritional and dietary supplements.

### SUMMARY OF THE INVENTION

In one embodiment, the present invention relates to a cosmetic and skin care formulation comprising: borojo; and at least one humectant; and at least one emollient. In another embodiment, the formulation further comprises peptides. In yet another embodiment, the formulation further comprises anti-oxidants. In still another embodiment, the formulation further comprises vitamins and minerals.

In a further embodiment, the humectant is selected from a group consisting essentially of glycerin, glycols, butylene glycol, propylene glycol, pentylene glycol, polyquarternium-7, and extracts thereof and mixtures thereof.

In still a further embodiment, the emollient is selected from a group consisting essentially of sweet almond oil, Squalene, apricot oil, safflower oil, sunflower oil, oils and mixtures thereof.

In yet a further embodiment, the formulation further comprises an anti-aging agent. In still yet a further embodiment, the formulation further comprises at least one exfoliating agent. In another further embodiment, the exfoliating agent is selected from a group consisting essentially of pearl, rice, polymers and mixtures thereof. In yet another further embodiment, the formulation is used as a cleanser. In still another further embodiment, the formulation is topically applied to the user's skin. In still yet another further embodiment, the formulation is in a form selected from a group consisting essentially of creams, serums, gels, ointments, lotions, powders and fluids.

In another embodiment, the present invention relates to a formulation comprising borojo wherein said formulation is topically applied to a user's skin.

In still another embodiment, the present invention relates to a formulation comprising: borojo; at least one mineral; at least one vitamin and at least one protein. In yet another embodiment, the formulation further comprises peptides. In still yet another embodiment, the formulation further comprises anti-oxidants.

In a further embodiment, the formulation is used as a nutritional supplement. In still a further embodiment, the formulation is used as dietary and/or nutritional supplements. In yet a further embodiment, the formulation is encapsulated. In still yet a further embodiment, the formulation functions as a carrier of nutrients. In another embodiment, the formulation may be incorporated into foodstuff, beverages, drinks and other consumables such as bars, etc.

### DETAILED DESCRIPTION OF THE INVENTION

As required, detailed embodiments of the present invention are disclosed herein; however, it is to be understood that the disclosed embodiments are merely exemplary of the invention that may be embodied in various forms. Therefore, specific functional details disclosed herein are not to be interpreted as limiting, but merely as a basis for the claims and as a representative basis for teaching one skilled in the art to employ the present invention in various ways.

The pulp of this fruit has a high content of carbohydrates, fiber, calcium and phosphorus. Borojo has one of the highest contents of phosphorus from all known fruits (160 mg/100 g of pulp) and one of the highest levels of water soluble B vitamins. In fact it is highly energetic with soluble solids (30° Brix) consisting mainly of fructose, glucose and proteins with apparently non limiting amounts of essential amino acids. Borojo has a high content of amino acids, a pound of this fruit is the equivalent of three pounds of fresh meat. The indigenous people of Columbia and in South/Central America believe it has a number of beneficial properties that include:

Stabilizes blood sugar levels

Natural aphrodisiac

High phosphorus content stimulates cellular activity promoting hormone regenerations

Helps to maintain normal blood pressure

Fortifies DNA and RNA structure and helps cellular regeneration

High energy source

Helps with cholesterol problems

Borojo is used by many people in the belief that it prevents, treats or relieves the symptoms of the following diseases: Cancer, Diabetes, Alzheimer's, Parkinson's and HIV.

Borojo is also claimed to have the following benefits:

Rejuvenation

Improves short term memory

Regulates menstrual cycles

Regenerates skin and hair

Boosts cell immunity

Anti-depression, anti-stress and anti-hangover

Aids in rehabilitation process from addictions

Energizing

The indigenous peoples throughout Columbia have long believed that Borojo has preservative properties. This belief can be seen in their use of Borojo to embalm cadavers, a practice which continues to this day. Many indigenous peoples believe that Borojo also has curative properties which when applied to wounds aid in their healing. Borojo is also used by native in Columbia to treat burns. The natives boil the Borojo until is becomes a sticky and tar-like consistency. Once the desired consistency is achieved, the boiled fruit is applied to the burn to promote healing.

Borojo is a good source of protein, vitamins and minerals, such as calcium and phosphorus. The fruit has a high and significant polyphenol content. Polyphenols are contained in many fruits and teas. Polyphenols are attributed to many of the health benefits of such foods as green tea and pomegranates. Through scientific analysis it has been discovered that unlike most fruits and teas, Borojo has only one major polyphenol compound. It appears that Borojo may contain a new type of polyphenol compound that has never been seen before in any other plant.

In a further embodiment, the formulation may further comprise an herbal extract selected from a group consisting essentially of angelica extract, ashwaganda extract, astragalus extract, bilberry extract, black cohosh extract, blue cohosh extract, blessed thistle extract, burdock extract, California poppy extract, cactus extract, celandine extract, chamomile extract, chaparral extract, chaste tree extract, chickweed extract, cleavers extract, coltsfoot extract, corn silk extract, cramp bark extract, damiana extract, dandelion extract, devil's claw extract, dong quai extract, echinacea extract, sib ginseng extract, eyebright extract, false unicorn root extract, fenugreek extract, ginkgo extract, ginseng root extract, goldenrod extract, goldenseal extract, gotu kola extract, horsetail extract, hyssop extract, kava kava extract, lady's mantle extract, licorice root extract, milk thistle extract, motherwort extract, nettle extract, oats extract, Oregon grape root extract, osha root extract, passionflower extract, pau d'arco extract, plantain extract, red clover extract, red root extract, reishi mushroom extract, sarsaparilla extract, saw palmetto extract, St. John's wort extract, schisandra berry extract, shepherds purse extract, skullcap extract, slippery elm extract, usnea lichen extract, uva ursi extract, valerian extract, wild cherry extract, wild yam extract, wormwood extract, yellow dock extract, yucca root extract and mixtures thereof. In a further embodiment, the herbal extract of the present invention also uses botanical extracts.

In still a further embodiment, the formulation comprising an aromatherapeutical substance which comprises essential oils and the essential oils can be selected from a group consisting essentially of absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemon oil, lemongrass oil, lemon eucalyptus oil, lemon myrtle oil, lemon 5 fold oil, lican oil, lime oil, litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetivewr oil, wintergreen oil, wild verbena oil, wormwood oil, yarrow oil, ylang ylang oil and mixtures thereof.

In another further embodiment, the formulation further comprises vitamins, minerals, peptides, anti-oxidants, anti-aging agents, humectants and/or emollients. In still a further embodiment, the humectant is selected from a group consisting essentially of glycerin, glycols, butylene glycol, propylene glycol, pentylene glycol, polyquarternium-7, and extracts thereof and mixtures thereof. In yet a further embodiment, the emollient is selected from a group consisting essentially of sweet almond oil, Squalene, apricot oil, safflower oil, sunflower oil, oils and mixtures thereof.

In another further embodiment, the formulation further comprises vitamins, minerals, peptides, anti-oxidants, anti-aging agents, humectants and/or emollients. In still a further embodiment, the humectant is selected from a group consisting essentially of glycerin, glycols, butylene glycol, propylene glycol, pentylene glycol, polyquarternium-7, and extracts thereof and mixtures thereof. In yet a further embodiment, the emollient is selected from a group consisting essentially of sweet almond oil, Squalene, apricot oil, safflower oil, sunflower oil, oils and mixtures thereof.

In one embodiment, the formulations of the present invention do not have iron, copper or manganese, because these trace minerals are known to interact with vitamin C to generate free radicals that are harmful to our body. These formulations do not have herbal products, because some of them are known to interact with prescription and non-prescription medications to produce adverse effects. These formulations do not have heavy metals such vanadium, zirconium, and molybdenum, because they are known to be neurotoxic. Our body does not have any significant way of removing these heavy metals from the tissue.

Therefore, while the embodiments of the present invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A cosmetic and skin care formulation comprising: borojo; and at least one humectant; and at least one emollient.

2. The formulation of claim 1 further comprising peptides.

3. The formulation of claim 1 further comprising anti-oxidants.

4. The formulation of claim 1 further comprising vitamins and minerals.

5. The formulation of claim 1 wherein said humectant is selected from a group consisting essentially of glycerin, glycols, butylene glycol, propylene glycol, pentylene glycol, polyquarternium-7, and extracts thereof and mixtures thereof.

6. The formulation of claim 7 wherein said emollient is selected from a group consisting essentially of sweet almond oil, Squalene, apricot oil, safflower oil, sunflower oil, oils and mixtures thereof.

7. The formulation of claim 1 further comprising an anti-aging agent.

8. The formulation of claim 1 further comprising at least one exfoliating agent.

9. The formulation of claim 8 wherein said exfoliating agent is selected from a group consisting essentially of pearl, rice, polymers and mixtures thereof.

10. The formulation of claim 1 wherein said formulation is used as a cleanser.

11. The formulation of claim 1 wherein said formulation is topically applied to the user's skin.

12. The formulation of claim 1 wherein said formulation is in a form selected from a group consisting essentially of creams, seruns, gels, ointments, lotions, powders and fluids.

13. A formulation comprising borojo wherein said formulation is topically applied to a user's skin.

14. A formulation comprising: borojo; at least one mineral; at least one vitamin and at least one protein.

15. The formulation of claim 14 further comprising peptides.

16. The formulation of claim 14 further comprising anti-oxidants.

17. The formulation of claim 14 wherein said formulation is used as a nutritional supplement.

18. The formulation of claim 14 wherein said formulation is used as a dietary supplement.

19. The formulation of claim 14 wherein said formulation is encapsulated.

20. The formulation of claim 14 wherein said formulation functions as a carrier of nutrients.
